(19) United States
(12) Patent Application Publication
(10) Pub. No.: US 2023/0243754 A1
(43) Pub. Date: Aug. 3, 2023

(54) ELECTRONIC DEVICE FOR ANALYZING AN ANALYTE PRESENT IN A FLUID AND CONSUMABLE AND INTERCHANGEABLE SENSOR, METHOD OF MANUFACTURING SAID DEVICE AND SAID CONSUMABLE AND INTERCHANGEABLE SENSOR

(71) Applicant: ARYBALLE, GRENOBLE (FR)

(72) Inventor: Thierry CHAVIGNER, GRENOBLE (FR)

(21) Appl. No.: 18/009,763

(22) PCT Filed: Jun. 14, 2021
(86) PCT No.: PCT/FR2021/051062

§ 371 Date: Dec. 12, 2022

(30) Foreign Application Priority Data
Jun. 12, 2020 (FR) 20 06163

(51) Int. Cl. G01N 21/77; G01N 29/12

(52) U.S. Cl. CPC G01N 21/7703; G01N 29/12; G01N 2291/023; G01N 2021/7779

(57) ABSTRACT

The invention relates to an electronic device for analyzing an analyte (2) present in a fluid, comprising: a consumable and interchangeable sensor (10) comprising temporary receptors (14) capable of an interaction with the analyte present in the fluid, causing a change in local property; a sensor holder (50) in which the sensor is intended to be reversibly placed; and a transducer for the change in local property (130, 131; 230, 231), positioned on the sensor and/or on the sensor holder and able to convert the change in local property into an electronic signal expressing the change in local property. The sensor comprises a protection (17) for the temporary receptors. The invention also relates to the method of manufacturing this device, as well as to the consumable and interchangeable sensor and to its method of manufacturing.

### TECHNICAL FIELD

The field of the invention is that of techniques for measurement and physical analysis, in particular electronic.

The invention relates more particularly to an electronic device for analyzing an analyte present in a fluid and to the method of manufacturing this device. The invention also relates to a sub-assembly of this device, namely a consumable and interchangeable sensor, and to its manufacturing method.

### PRIOR ART

Electronic analysis devices are known that enable detecting the presence of an analyte in a fluid such as a gas or a liquid, identifying it, and possibly measuring its concentration in the fluid. The analyte may be a combination of target compounds, for example a mixture of VOCs (Volatile Organic Compounds) which can cause an odor. For this purpose, these devices are sometimes classified as electronic noses or tongues, depending on whether they work for gases or for liquids. The principle of detection in these devices may be based on interactions between the analyte and receptors integrated into a sensor. The interactions are based on properties of physicochemical affinity between the receptors and the analyte, and in particular between the receptors and the target compounds of the analyte. These interactions cause a change in one or more local properties indicative of the presence of the analyte, or even of the amount of analyte present.

The receptors can be selected among various compounds or materials suitable for forming a temporary ligand for the target compounds. A non-exhaustive list includes: some specific molecules, some peptides, some polymers, some biomarkers, some nanoparticles, some carbon nanotubes. The binding forces involved are generally weak forces (Van Der Waals type).

A transducer is generally used to convert this change in local property into a multidimensional electronic signal representative of this change in local property. An electronic signal is generated for each receptor. The set of electronic signals constitutes the multidimensional electronic signal. By processing and analyzing this multidimensional electronic signal, it is then possible to make a qualitative or even quantitative determination of the analyte present in the analyzed fluid.

Often, the receptors used to interact with the analyte are temporary receptors, meaning they have a shorter lifespan than the other components of the device. Indeed, interaction between the receptors and the analyte can lead to saturation of the receptors. Once the receptors are saturated, they no longer can interact properly with the analyte in the fluid. The sensor incorporating the receptors must then be replaced.

The receptors may also be specific for the type of analyte one wishes to detect. Indeed, the receptors may have a particular affinity for a given type of analyte. If one wishes to change the type of analyte to be detected, it may thus be necessary to replace the sensor with another sensor having receptors suitable for detecting the new type of analyte one wishes to detect.

It is therefore understood that to limit costs, it is preferable to be able to replace the sensor in the device rather than having to change the entire device, when the temporary receptors are no longer working or when one wishes to detect another analyte.

Patent WO2006/113177 already proposes a device in which the sensor is removably positioned in a base unit. When necessary, it is possible to change the sensor while retaining the other components of the device.

However, the device according to WO2006/113177 is not suitable for sensors comprising receptors sensitive to atmospheric contamination prior to their integration into the sensor of the device. Such contaminations can degrade these receptors. In particular, molecules contained in the air, for example, can interact with the receptors. The receptors can then be saturated even before they are positioned in the device, and therefore even before they have interacted with an analyte present in the fluid to be analyzed.

There is therefore a need for an electronic analysis device in which the sensor can be replaced by a new sensor and which guarantees the quality of the receptors of the new sensor which is inserted into the device.

In this context, the invention aims to satisfy at least one of the following objectives:

one objective of the invention is to provide an electronic analysis device having an easily replaceable sensor,
another objective of the invention is to provide an electronic analysis device guaranteeing the quality of the receptors of the sensor installed in the device,
another objective of the invention is to provide an electronic analysis device making it possible to reduce production costs,
another objective of the invention is to provide an electronic analysis device adaptable to different fields of use,
another objective of the invention is to provide a sensor that is easy to handle, meaning it can be handled without any risk of damaging the receptors integrated into the sensor,
another objective of the invention is to provide a sensor whose receptors are protected from external contamination prior to its placement in the device.

### SUMMARY

The above objectives, among others, are achieved by the invention which, according to a first aspect, relates to an electronic device for analyzing an analyte present in a fluid, comprising:

A. a consumable and interchangeable sensor comprising an electronic chip and a cover integrally secured to the electronic chip, the electronic chip comprising a measurement chamber comprising temporary receptors capable of an interaction with the analyte present in the fluid, the interaction causing a change in local property, and the cover comprising an opening suitable for admitting the fluid into the measurement chamber and for discharging the fluid from the measurement chamber;
B. a sensor holder comprising a housing in which the sensor is intended to be reversibly placed;
C. a transducer for the change in local property caused by the interaction between the temporary receptors and the analyte; this transducer being:

able to convert the change in local property into an electronic signal expressing the change in local property; and,

positioned on the sensor and/or on the sensor holder,

the sensor comprising a protection for the temporary receptors which is configured to be active prior to placement of the sensor in the housing of the sensor holder and configured to cooperate with the sensor holder so as to be deactivated by placement of the sensor in the housing of the sensor holder.

The heart of the invention consists of the development of a preliminary protection for the temporary receptors forming the sensitive portion of the device. The protection for the temporary receptors makes it possible to protect the temporary receptors by isolating them from the outside atmosphere. The temporary receptors are therefore not altered prior to the sensor being placed in the sensor holder. In other words, they are not altered prior to being used in the device. This protection is put in place just after manufacture, and is therefore in place during storage of the sensor and until the sensor is integrated into the electronic analysis device where the temporary receptors no longer risk exposure to exogenous pollution. Protection of the temporary receptors is obtained without interfering with the ease of placement and removal of the sensor in/from the device, or with the quality of the analysis.

In addition, when the sensor is seated in the housing of the sensor holder, the cooperation between the protection for the temporary receptors and the sensor holder makes it possible to deactivate the protection for the temporary receptors. The fluid can then circulate in the measurement chamber of the sensor, and reach the temporary receptors. Deactivation of the protection thus guarantees optimal use of the sensor and of the temporary receptors.

In addition, because the sensor comprising the temporary receptors is reversibly inserted into the sensor holder, when the temporary receptors no longer function or when one wishes to analyze another analyte, it is possible to change the sensor while retaining the other components of the device, in particular the sensor holder and all or part of the transducer.

According to a variant of the invention, the temporary receptors in the measurement chamber are selected among molecules, peptides, polymers, biomarkers, carbon nanotubes or nanoparticles.

According to a variant of the invention, the analyte is a combination of target compounds, for example volatile organic compounds, contained in the fluid.

According to one example, the analyte is a mixture of VOCs (volatile organic compounds) that is characteristic of an odor contained in the fluid.

According to a variant of the invention, the protection for the temporary receptors comprises a protective wrapper configured for:

closing off the opening of the cover prior to placement of the sensor in the housing of the sensor holder, and
cooperating with the sensor holder so as to be pierced facing the opening of the cover when the sensor is placed in the housing of the sensor holder.

When appropriate, the protective wrapper may be

a polymer film, for example a polyolefin, optionally a substituted one such as polytetrafluoroethylene (PTFE) or a silicone such as polydimethylsiloxane (PDMS); and/or
a film having a thickness comprised between 50 μm and 150 μm.

In one example, the opening of the cover has an inlet opening configured to admit fluid into the measurement chamber and an outlet opening configured to discharge fluid from the measurement chamber. Preferably, the protective wrapper comprises a first cap configured to close off the inlet opening prior to placement of the sensor in the housing of the sensor holder and a second cap configured to close off the outlet opening prior to placement of the sensor in the housing of the sensor holder.

According to a variant of the invention, the change in local property is a change of optical index in the sensor or a change of mass of a membrane in the sensor.

According to a first variant of the invention, the electronic chip is a photonic chip comprising at least one light guide in which the temporary receptors are arranged, the light guide comprising a light input and a light output, and the transducer comprises:

a coherent light source aligned with the light input of the light guide and capable of emitting a beam of coherent light into the light guide of the electronic chip;
an optical detector aligned with the light output of the light guide and able to measure an optical parameter of the beam of coherent light exiting the light guide.

When there are such, the coherent light source and the optical detector may be positioned on the sensor holder. Alternatively, the coherent light source and the optical detector may be positioned on the photonic chip.

According to a second variant of the invention, the electronic chip is an electromechanical chip comprising a membrane on which the temporary receptors are arranged, and the transducer comprises an actuator capable of causing the membrane to vibrate, and a vibration frequency detector for detecting the vibration frequency of the membrane.

When there are such, the actuator and the vibration frequency detector may be positioned on the sensor holder. Alternatively, the actuator and the vibration frequency detector may be positioned on the electromechanical chip.

According to a variant of the invention, the device has the following characteristics:

the housing of the sensor support is delimited by a bottom and side walls;
the cover comprises, on its largest external face, at least one inlet opening and at least one outlet opening, at least one of these openings preferably being constituted by a pipe projecting with respect to this external face ;
the sensor is capable of cooperating, for its installation in the support and its removal from the support, with the housing of the support, by interlocking and sliding, i.e. by a sliding effect, and
this interlocking/sliding takes place according to an arrangement in which the openings of the cover are opposite the bottom of the housing.

According to a variant of the invention, the sensor holder comprises:

a positioning guide for the sensor, configured to guide the sensor as it is placed in the housing of the sensor holder;
a retaining member for holding the sensor in the housing, configured to hold the sensor in the housing when the sensor is placed in the housing of the sensor holder;
a connection socket configured to cooperate with the opening of the cover when the sensor is placed in the housing.

According to a variant of the invention, the retaining member comprises a bimetallic spring or is formed by a lid attached to the sensor holder and configured to encapsulate the sensor.

According to a variant of the invention, the opening of the cover and the connection socket are configured to allow the protective wrapper to be pierced facing the opening of the cover.

According to a variant of the invention, the opening of the cover has an inlet opening configured to admit fluid into the measurement chamber and an outlet opening configured to discharge fluid from the measurement chamber, the connection socket of the sensor holder comprises an inlet connection socket configured to cooperate with the inlet opening of the cover and an outlet connection socket configured to cooperate with the outlet opening of the cover after placement of the sensor in the housing of the sensor holder,

the sensor holder comprises an inlet duct for fluid and an outlet duct for fluid, the inlet connection socket of the sensor holder communicating with the inlet duct of the sensor holder and the outlet connection socket of the sensor holder communicating with the outlet duct of the sensor holder.

According to a variant of the invention, the inlet opening and the outlet opening of the cover are each a pipe projecting from the cover;

the inlet connection socket and the outlet connection socket of the sensor holder are each a cavity having a bottom, the inlet duct and outlet duct respectively extending, preferably axially, from the bottom from one end;
and each projecting pipe has a shape and dimensions complementary to those of the end of the inlet duct or outlet duct, so as to allow fitting each pipe into the end of the corresponding inlet duct or outlet duct.

According to a variant of the invention, the sensor holder comprises an upper face comprising a recess forming the housing.

According to a variant of the invention, the upper face of the sensor holder is flat and the recess is defined by inclined side walls forming an angle, strictly greater than 85° and strictly less than 90°, with the flat upper face of the sensor holder.

According to a variant of the invention, the sensor is protruding relative to the sensor holder when the sensor is placed in the housing.

According to a second aspect of the invention, a consumable and interchangeable sensor is provided, comprising an electronic chip and a cover integrally secured to the electronic chip,

this electronic chip comprising a measurement chamber comprising temporary receptors capable of an interaction with an analyte present in a fluid to be analyzed, the interaction causing a change in local property, and the cover comprising an opening suitable for admitting the fluid into the measurement chamber and for discharging the fluid from the measurement chamber,
the sensor being intended to be reversibly placed in a housing of a sensor holder, and optionally comprising all or part of a transducer for the change in local property caused by the interaction between the temporary receptors and the analyte, the transducer being able to convert the change in local property into an electronic signal expressing the change in local property, and
the sensor comprises a protection for the temporary receptors which is configured to be active prior to placement of the sensor in the housing of the sensor holder and configured to cooperate with the sensor holder so as to be deactivated by placement of the sensor in the housing of the sensor holder; this protection comprising a protective cover configured to:

closing off the opening of the cover prior to placement of the sensor in the housing of the sensor holder, and

cooperating with the sensor holder so as to be pierced facing the opening of the cover when the sensor is placed in the housing of the sensor holder.

According to a variant of the invention, the protective wrapper is:

a polymer film, for example a polyolefin, optionally a substituted one such as polytetrafluoroethylene (PTFE) or a silicone such as polydimethylsiloxane (PDMS); and/or
a film having a thickness comprised between 50 μm and 150 μm.

According to a variant of the invention, the opening of the cover has an inlet opening configured to admit fluid into the measurement chamber and an outlet opening configured to discharge fluid from the measurement chamber. Preferably, the protective wrapper comprises a first cap configured to close off the inlet opening prior to placement of the sensor in the housing of the sensor holder and a second cap configured to close off the outlet opening prior to placement of the sensor in the housing of the sensor holder.

According to a variant of the invention, the change in local property is a change of optical index in the sensor or a change of mass of a membrane in the sensor.

According to a variant of the invention, the electronic chip is a photonic chip comprising at least one light guide in which the temporary receptors are arranged, the light guide comprising a light input and a light output, and the transducer comprises:

a coherent light source aligned with the light input of the light guide and capable of emitting a beam of coherent light into the light guide of the electronic chip;
an optical detector aligned with the light output of the light guide and able to measure an optical parameter of the beam of coherent light at the outlet of the light guide.

According to a variant of the invention, the coherent light source and the optical detector are positioned on the sensor holder. Alternatively, the coherent light source and the optical detector may be positioned on the photonic chip.

According to a variant of the invention, the electronic chip is an electromechanical chip comprising a membrane on which the temporary receptors are arranged, and the transducer comprises an actuator capable of causing the membrane to vibrate.

According to a third aspect of the invention, a method is provided for manufacturing a consumable and interchangeable sensor according to the invention, essentially consisting of:

i) preparing a silicon wafer;
ii) functionalizing the silicon wafer to form a plurality of electronic chips;
iii) introducing temporary receptors onto a surface of the silicon wafer, on each electronic chip, to form measurement chambers;
iv) covering, with a protective layer, the surface of the silicon wafer onto which the temporary receptors have been introduced, the protective layer forming a cover for each measurement chamber;
v) providing an opening on each cover to allow admitting a fluid into the measurement chamber associated with the cover and discharging the fluid from the measurement chamber associated with the cover;
vi) cutting the silicon wafer to separate each assembly formed by a measurement chamber and a cover;
vii) providing a protection on the opening of each cover so as to form the sensors;
viii) collecting the sensors.

According to a fourth aspect of the invention, a method is provided for manufacturing an electronic analysis device according to the invention, essentially consisting of:

i) mass-producing consumable sensors;
ii) preferably, storing the mass-produced consumable sensors;
iii) separately producing sensor holders;
iv) assembling a consumable sensor with a sensor holder, preferably on a to-order basis.

According to a variant of the invention, the assembly step (iv) essentially consists of:

iv.1) taking a consumable sensor;
iv.2) preparing a sensor holder;
iv.3) positioning the consumable sensor relative to the sensor holder so that the opening of the cover is aligned with the connection socket of the sensor holder;
iv.4) reversibly placing the sensor in the housing of the sensor holder;
iv.5) and, simultaneously or not simultaneously with step (iv.4), piercing the protective wrapper facing the opening of the cover.

Throughout the present disclosure, any use of the singular indiscriminately designates a singular or a plural.

### BRIEF DESCRIPTION OF DRAWINGS

Other features, details and advantages will become apparent from reading the detailed description below, and from analyzing the accompanying drawings, in which:

FIG. 1 shows a consumable and interchangeable sensor comprising an electronic chip and a cover integrally secured to the electronic chip, as well as a light source and an optical detector which are positioned on the electronic chip;

FIG. 2 shows a side view of the consumable and interchangeable sensor of FIG. 1;

FIG. 3 shows a top view of the consumable and interchangeable sensor of FIG. 1, in which the cover and the optical detector have been removed to reveal a measurement chamber comprised in the electronic chip;

FIG. 4 schematically illustrates the consumable and interchangeable sensor of FIG. 1, prior to its placement on a sensor holder.

FIG. 5 schematically illustrates an electronic analysis device for analyzing an analyte present in a fluid, comprising the consumable and interchangeable sensor of FIG. 1 placed in a sensor holder;

FIG. 6 schematically illustrates an optical guide comprising temporary receptors which is capable of being integrated into the consumable and interchangeable sensor of FIG. 1;

FIG. 7 schematically illustrates a membrane on which are arranged temporary sensors which is capable of being integrated into the consumable and interchangeable sensor of FIG. 1;

FIG. 8 schematically illustrates the reaction between temporary receptors and an analyte to be analyzed;

FIG. 9 shows a diagram representative of the detection of an analyte by the analyte-analyzing electronic analysis device of FIG. 9.

FIG. 10 illustrates the method of manufacturing the consumable and interchangeable sensor of FIG. 1;

FIG. 11 illustrates the method of manufacturing an analyte-analyzing electronic analysis device of FIG. 6.

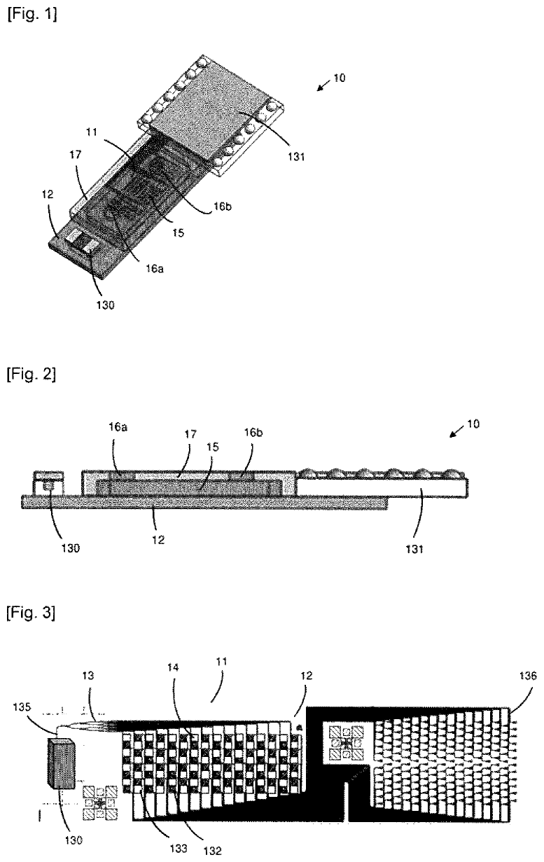

### DESCRIPTION OF EMBODIMENTS

In the figures, the same references designate identical or similar elements.

In FIGS. 1, 2, and 4, a consumable and interchangeable sensor 10 is represented. This sensor 10 makes it possible to reveal the presence of an analyte 2 in a fluid to be analyzed. The fluid may be a gas or a liquid. This analyte 2 may be a combination of target compounds, for example volatile organic compounds, contained in the fluid. In particular, the analyte 2 may be a mixture of volatile organic compounds that is characteristic of an odor contained in the fluid.

This sensor 10 comprises an electronic chip 12. The electronic chip 12 comprises a measurement chamber 11. This measurement chamber 11 comprises temporary receptors 14. These temporary receptors 14 can interact with the analyte 2 contained in the fluid to be analyze. The interaction between temporary receptors 14 and analyte 2 causes a change in local property. Thus, when the temporary receptors 14 are in the presence of the analyte 2, at least one local property characteristic of the environment in which the temporary receptors 14 are positioned is modified. The local property may be the optical index of the medium or a change in mass of a membrane 23 on which the temporary receptors 14 are located.

The temporary receptors 14 may be selected among molecules, peptides, polymers, biomarkers, carbon nanotubes or nanoparticles.

The sensor 10 also comprises a cover 15 integrally secured to the electronic chip 12. The cover 15 comprises an inlet opening 16a allowing the fluid to be analyzed to be admitted into the measurement chamber 11 and an outlet opening 16b allowing the fluid to be discharged from the measurement chamber 11. The fluid can thus circulate from the inlet opening 16a to the outlet opening 16b. The inlet opening 16a and the outlet opening 16b are each formed by a pipe projecting from the cover 15.

The inlet opening 16a is positioned near a first end of the measurement chamber 11 and the outlet opening 16b is positioned near a second end of the measurement chamber 11, thus making it possible to ensure the passage of the fluid at the temporary receptors 14. In a variant not shown, the cover 15 could comprise a single opening which allows both admitting the fluid to be analyzed into the measurement chamber 11 and discharging the fluid from the measurement chamber 11.

The sensor 10 also comprises a protection 17 for the temporary receptors 14. This protection 17 makes it possible to protect the temporary receptors 14 from fluid, and in particular from the air surrounding the sensor 10, by isolating the temporary receptors 14 from the outside.

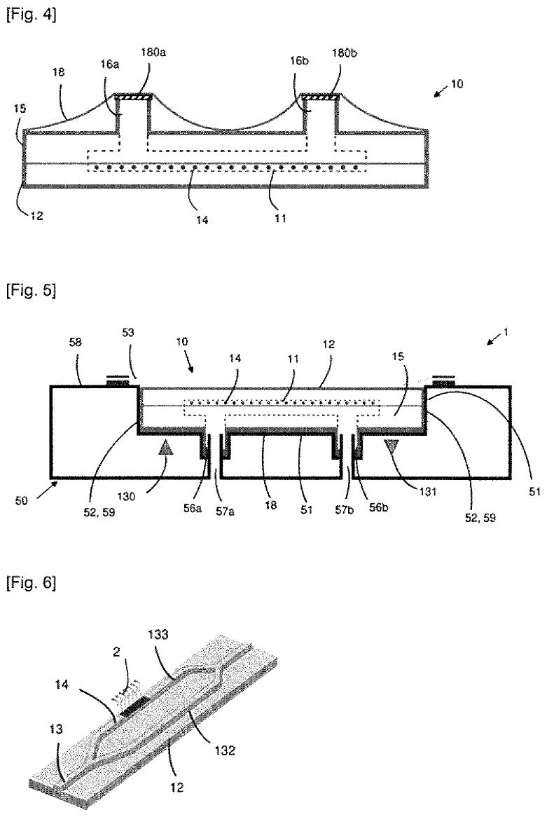

In the variant shown in FIGS. 4 and 5, the protection 17 for the temporary receptors 14 comprises a film forming a protective wrapper 18. As can be seen in FIG. 4, the protective wrapper 18 closes off the inlet opening 16a and outlet opening 16b of the cover 15. The temporary receptors 14 are then no longer in contact with the outside air via the inlet opening 16a and outlet opening 16b. The protective wrapper 18 also comprises a first cap 180a closing off the inlet opening 16a and a second cap 180b closing off the outlet opening 16b. The first cap 180a and second cap 180b allow reinforcing the action of the film at the inlet opening 16a and outlet opening 16b, to prevent any passage of fluid through the inlet opening 16a and outlet opening 16b.

The protective wrapper 18 may be formed by a polymeric material, for example a polyolefin, optionally a substituted one such as polytetrafluoroethylene (PTFE) or a silicone such as polydimethylsiloxane (PDMS). In particular, the material is deformable, can be made into a film, is stretchable, is waterproof, and must not release volatile organic compounds (VOCs). For example, the amount of volatile organic compounds released is less than 0.1 μg/g.

The protective wrapper 18 has a thickness comprised between 50 μm and 150 μm. The first cap 180a and the second cap 180b have a thickness comprised between 50 μm and 100 μm.

FIG. 5 shows an electronic analysis device 1 comprising the sensor 10. The electronic analysis device 1 makes it possible to analyze the analyte 2 whose presence in the fluid to be analyzed is revealed by the sensor 10. It thus makes it possible to detect the presence of the analyte 2 in the fluid to be analyzed, or even to determine the amount of analyte 2 in the fluid to be analyzed.

The electronic analysis device 1 also includes a sensor holder 50. The sensor holder 50 comprises a housing 51 into which the sensor 10 is reversibly placed. The sensor 10 can thus be removed from the sensor holder 50 without damaging the electronic analysis device 1 and the sensor holder 50.

The sensor holder 50 comprises an upper face 58 comprising a recess forming the housing 51. The upper face 58 is flat and the recess is defined by inclined side walls 59 of the housing 51. For example, the side walls 59 form an angle strictly greater than 85° and strictly less than 90°, preferably an angle of 89°, with the flat upper surface 58.

As can be seen in FIG. 4, prior to placement of the sensor 10 in the housing 51 of the sensor holder 50, the protective wrapper 18 closes off the inlet opening 16a and outlet opening. 16b. As can be seen in FIG. 5, when the sensor 10 is placed in the housing 51 of the sensor holder 50, the protective wrapper 18 cooperates with the sensor holder 50. The protective wrapper 18 gets perforated face to the inlet opening 16a and outlet opening 16a of the cover 15. In the event that the cover 15 only comprises a single opening, the protective wrapper 18 would get perforated face to this opening. The fluid to be analyzed can then flow within the sensor 10 and come into contact with the temporary receptors 14.

The protective wrapper 18 therefore is only pierced when the sensor 10 is placed in the sensor holder 50. The sensor 10 can therefore only be used once it has been installed in the electronic analysis device 1, which guarantees the quality of the temporary receptors 14 of the installed sensor 10.

In order to properly place the sensor 10 in the housing 51, the sensor holder 50 comprises a positioning guide 52 for the sensor 10 in order to guide the sensor as it is placed in the housing 51 of the sensor holder 50. The positioning guide 52 is formed by the side walls 59 of the housing 51. The side walls 59 come into contact with the side walls of the sensor 10 and thus guide the sensor 10.

The sensor holder 50 also comprises a retaining member 53 for holding the sensor 10 in the housing 51 in order to hold the sensor 10 in the housing 51 when the sensor 10 is placed in the housing 51 of the sensor holder 50. The retaining member 53 comprises a bimetallic spring. The bimetallic spring is movable so as to allow the sensor 10 to pass through when it is placed in the housing 51, and so as to be positioned against the sensor 10 once the sensor is in place in the housing 51, thus preventing the sensor 10 from leaving the housing 51. When one wishes to remove the sensor 10 from the housing 51, the retaining member 53 can once again be moved to allow the sensor 10 to come out. The retaining member 53 thus forms a reversible retention of the sensor 10.

The sensor holder 50 also has an inlet connection socket 56a and an outlet connection socket 56b. The inlet connection socket 56a and the outlet connection socket 56b of the sensor holder 50 are each a cavity having a bottom. When the sensor 10 is placed in the housing 51, the inlet opening 16a is inserted into the inlet connection socket 56a. The inlet connection socket 56a then allows the protective wrapper 18 and the first cap 180a to get perforated face to the intake opening 16a. Similarly, when the sensor 10 is placed in the housing 51, the outlet opening 16b is inserted into the outlet connection socket 56b. The outlet connection socket 56b then allows the protective wrapper 18 and the second cap 180b to get perforated face to the outlet opening 16b.

The sensor holder 50 also comprises an inlet duct 57a for fluid and an outlet duct 57b for fluid. The inlet connection socket 56a of the sensor holder 50 communicates with the inlet duct 57a of the sensor holder 50 and the outlet connection socket 56b of the sensor holder 50 communicates with the outlet duct 57b of the sensor holder 50. The inlet duct 57a and outlet duct 57b each extend axially from one end, starting respectively from the bottom of the cavity forming connection socket 56a and from the bottom of the cavity forming outlet socket 56b.

Each projecting pipe forming the inlet opening 16a and outlet opening 16b has a shape and dimensions complementary to those of the end of the inlet duct 57a or outlet duct 57b, so as to allow the each pipe to fit onto the end of the corresponding inlet duct 57a or outlet duct 57b.

The electronic analysis device 1 also comprises a transducer for the change in local property caused by the interaction between the temporary receptors 14 and the analyte 2. This transducer makes it possible to convert the change in local property into an electronic signal expressing the change in local property.

In a first example, the transducer comprises a coherent light source 130 and an optical detector 131. The light source 130 may for example be a laser diode. The light source 130 and the optical detector 131 may be positioned on the sensor 10, as shown in FIGS. 1 and 2. Alternatively, they may be positioned on the sensor holder 50 as shown in FIG. 5. In another alternative, not shown, the light source 130 could be positioned on one among the sensor 10 and sensor holder 50 and the detector could be positioned on the other among the sensor 10 and sensor holder 50.

In this first example, the electronic chip 12 is a photonic chip comprising at least one light guide 13. The light guide has a light input 135 and a light output 136. The coherent light source 130 is aligned with the light input 135 so that the light source 130 can emit a beam of coherent light into the light guide 13. The optical detector 131 is aligned with the light output 136 so that the optical detector 131 can detect an optical parameter of the beam of coherent light exiting the light guide 130.

The light guide 13 is divided into a plurality of branches, and in each branch, the light guide 130 is again divided into two arms. A branch thus divided into two arms is shown in FIG. 6. Each branch of the light guide 130 thus comprises a reference arm 132 in which part of the light beam emitted by the light source 130 is guided by total internal reflection and a measurement arm 133 in which another part of the light beam emitted by the light source 130 is guided by total internal reflection and in which the temporary receptors 14 are arranged. The reference arm 132 and the measurement arm 133 are then merged back together, making it possible to recombine the light beam guided in the reference arm 132 and the light beam guided in the measurement arm 133.

Each branch of the light guide 130 forms an interferometer for detecting the presence of an analyte 2 in a fluid. In fact, when the fluid enters the measurement chamber 11, the temporary receptors 14 present in each measurement arm 133 of the branches of the light guide 13 will interact with the analyte 2. As can be seen in FIG. 8, the analyte 2 will, for example, cling to the temporary receptors 14. The interaction between the temporary receptors 14 and the analyte 2 will then change the optical index in the measurement arm 133. This change of optical index in the measurement arm 133 will generate a phase delay in the light beam guided in the measurement arm 133 while the phase of the light beam guided in the reference arm is unchanged. When the light beam exiting the measurement arm 133 and the light beam exiting the reference arm 132 are recombined, they form specific interference from the phase delay in the light beam guided in the measurement arm 133. Such interference is responsible for a specific light intensity distribution. This specific light intensity distribution is then detected by the optical detector 131. The optical detector 131 then transforms the specific light intensity distribution received into an electronic signal expressing the change in optical index, and therefore the interaction between the analyte 2 and the temporary receptors 14.

In particular, for each branch of the optical guide 13, the optical detector 131 receives a specific light intensity distribution. The optical detector 131 then transforms each of these specific light intensity distributions received into an electronic signal, expressing the change in optical index in the measurement arm 133 of the corresponding measurement branch, and therefore the interaction between the analyte 2 and the temporary receptors 14 in the corresponding branch. The set of electronic signals generated then forms a multidimensional electronic signal 31.

Thus, by means of the light source 130, the optical guide 13, and the detector 131, it is possible to detect the change in optical index generated by the interaction between the analyte 2 and the temporary receptors 14, and therefore to detect the presence of the analyte 2 in the analyzed fluid.

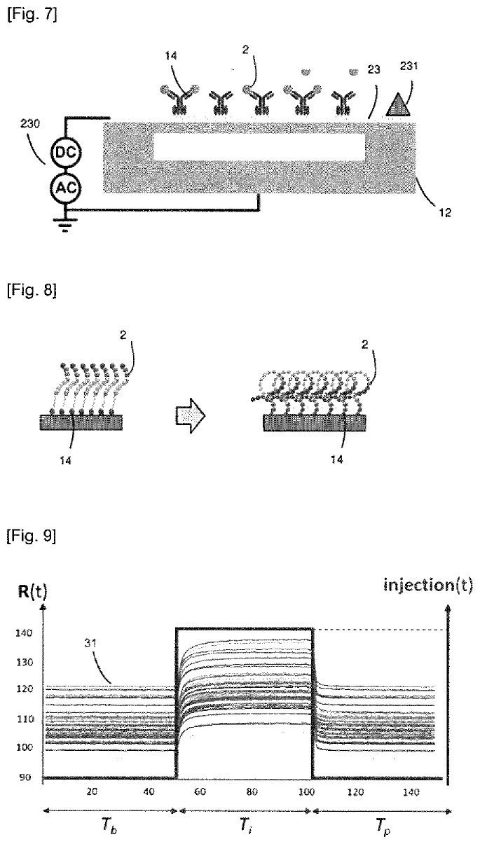

In a second example, shown in FIG. 7, the electronic chip 12 is an electromechanical chip. The electromechanical chip comprises a membrane 23.

The transducer comprises an actuator 230 which makes it possible to vibrate the membrane 23, and a vibration frequency detector 231. The actuator 230 and the vibration frequency detector 231 may be positioned on the sensor 10, as shown in FIG. 7. Alternatively, they could be positioned on the sensor holder 50. In another alternative, not shown, the actuator 230 could be positioned on one among the sensor 10 and sensor holder 50 and the vibration frequency detector 231 could be positioned on the other among the sensor 10 and sensor holder 50.

The temporary receptors 14 are arranged on the membrane 23. When fluid enters the measurement chamber 11, the temporary receptors 14 arranged on the membrane 23 will interact with the analyte 2. The analyte will for example attach to the temporary receptors 14. The interaction between the temporary receptors 14 and the analyte 2 will then change the mass of the membrane 23. This change in the mass of the membrane 23 will cause a change in its vibration frequency. This change in vibration frequency is specific to the change in mass of the diaphragm 23. When the diaphragm 23 is vibrated by the actuator 230, the vibration frequency detector 231 can thus detect this change in vibration frequency.

The vibration frequency detector 231 then transforms the change in vibration frequency into an electronic signal expressing the change in mass of the membrane 23, and therefore the interaction between the analyte 2 and the temporary receptors 14.

In particular, the electromechanical chip comprises a plurality of membranes 23 which can be vibrated by one or more actuators 230. And the vibration frequency detector 231 generates, for each membrane 23, an electronic signal expressing the change in mass of the corresponding membrane 23. The set of electronic signals generated then forms a multidimensional electronic signal 31.

Thus, by means of the actuator 230 and the vibration frequency detector 231, it is possible to detect the change in vibration frequency of the membrane 23, and therefore to detect the presence of the analyte 2 in the analyzed fluid.

According to one example, not shown, the electronic chip 12 comprises a reflecting film on which the temporary receptors 14 are arranged, and the transducer comprises a source of polarized light and an optical detector. The interaction between the analyte 2 and the temporary receptors 14 is responsible for a change of optical index in the measurement chamber 11. When polarized light is emitted from the light source, the proportion of light reflected by the reflective film varies depending on the optical index in the measurement chamber 11. The optical detector allows detecting the proportion of reflected light. The optical detector then transforms the proportion of reflected light into an electronic signal expressing the proportion of light reflected by the reflecting film, and therefore of the interaction between the analyte 2 and the temporary receptors 14. It is thus possible to detect the presence of the analyte 2 in the analyzed fluid.

In particular, the electronic chip 12 comprises a plurality of reflective films, on each of which are arranged the temporary receptors 14. And, for each reflective film, the optical detector generates an electronic signal expressing the proportion of light reflected by the corresponding reflective film. The set of electronic signals generated then forms a multidimensional electronic signal 31.

An example of a multidimensional electronic signal 31 generated by the optical detector 131 or the vibration frequency detector 231 is shown in FIG. 9.

During time period Tb, no fluid is introduced into the measurement chamber 11. The multidimensional electronic signal 31 has a baseline value. During time period Ti, the fluid to be analyzed is introduced into the measurement chamber 11. A change in the multidimensional electrical signal is then observed. This change is characteristic of the interaction between the analyte 2 and the temporary receptors 14. During period Tp, no fluid is introduced into the measurement chamber. The multidimensional electronic signal 31 returns to its baseline value. This time period Tp makes it possible to purge the measurement chamber 11 and also allows the analyte 2 that interacted with the temporary receptors 14 to exit the measurement chamber 11. At the end of period Tp, the measurement chamber 11 is then ready to receive a new fluid to be analyzed and the temporary receptors 14 are then ready to receive the analyte 2 of the new fluid to be analyzed.

However, it could be that a portion of the analyte 2 of the analyzed fluid remains on the temporary receptors 14. The multidimensional electronic signal does not return to its exact baseline value, but to a value close to this baseline value. If this value is too far from the baseline value, then the temporary receptors 14 must be changed. The sensor 10 must be replaced.

For example, the temporary receptors 14 can be tested before use, in ambient air. An initial baseline value for the multidimensional electronic signal 31 is then obtained. If during period Tp, the value of the multidimensional electronic signal 31 takes a value that differs by less than 10% from the initial baseline value, then the temporary receptors 14 can be retained, and the sensor 10 can be kept.

Conversely, if during time period Tp, the value of the multidimensional electronic signal 31 takes a value that differs by more than 10% from the initial baseline value, then the temporary receptors 14 must be changed, and the sensor 10 must be replaced.

The method of manufacturing the consumable and interchangeable sensor is shown in FIG. 10.

In a first step E1, a silicon wafer is prepared. In a second step E2, the silicon wafer is functionalized to form a plurality of electronic chips 12. The temporary receptors 14 are then introduced onto a surface of the silicon wafer, on each electronic chip 12, during a third step E3, to form measurement chambers 11. In a fourth step E4, the surface of the silicon wafer onto which the temporary receptors 14 were introduced is covered by a protective layer. The protective layer forms the cover 15 for each measurement chamber 11. An opening is then provided on each cover 15 in a fifth step E5, to allow admitting fluid into the measurement chamber 11 associated with the cover 15 and discharging fluid from the measurement chamber 11 associated with the cover 15. Then, in a sixth step E6, the silicon wafer is cut to separate each assembly formed by a measurement chamber 11 and cover 15. A protection is then provided on the opening of each cover 15 during a seventh step E7, so as to form the sensors 10. The sensors 10 are then collected during an eighth step E8.

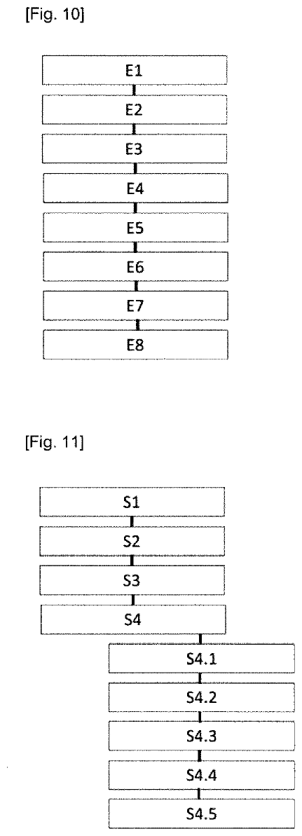

The method of manufacturing the electronic analysis device 1 is illustrated in FIG. 11.

In a first step S1, the consumable and interchangeable sensors are mass produced. In particular, they may be produced according to the method of manufacturing as described above. The sensors 10 are then preferably stored during a second step S2. In a third step S3, the sensor holders 50 are produced separately. Then, a consumable and interchangeable sensor is assembled with a sensor holder 50, preferably on a to-order basis, during a fourth step S4.

The fourth step S4 may in particular consist of taking a consumable and interchangeable sensor in a step S4.1, preparing a sensor holder 50 in a step S4.2, positioning the consumable sensor relative to the sensor holder so that the opening of the cover is aligned with the connection socket of the sensor holder during a step S4.3, reversibly placing the sensor in the housing of the sensor holder during a step S4.4, and, simultaneously or not simultaneously with step S4.4, piercing the protective wrapper facing the opening of the cover during a step S4.5.

1. Electronic device for analyzing (1) an analyte (2) present in a fluid, characterized in that it comprises:
A. a consumable and interchangeable sensor (10) comprising an electronic chip (12) and a cover (15) secured to the electronic chip, the electronic chip comprising a measurement chamber (11) comprising temporary receptors (14) capable of an interaction with the analyte present in the fluid, the interaction causing a change in local property, and the cover comprising an opening (16a, 16b) suitable for admitting the fluid into the measurement chamber and for discharging the fluid from the measurement chamber;
B. a sensor holder (50) comprising a housing (51) in which the sensor is intended to be reversibly placed;
C. a transducer for the change in local property (130, 131; 230, 231) caused by the interaction between the temporary receptors and the analyte; this transducer being:
able to convert the change in local property into an electronic signal expressing the change in local property; and,
positioned on the sensor and/or on the sensor holder,
in that the sensor comprises a protection (17) for the temporary receptors which is configured to be active prior to placement of the sensor in the housing of the sensor holder and configured to cooperate with the sensor holder so as to be deactivated by placement of the sensor in the housing of the sensor holder.

2. Device according to claim 1, wherein the temporary receptors of the measurement chamber are selected among molecules, peptides, polymers, biomarkers, nanoparticles or carbon nanotubes.

3. Device according to claim 1, wherein the analyte is a combination of target compounds, for example volatile organic compounds, contained in the fluid; preferably the analyte is a mixture of volatile organic compounds that is characteristic of an odor contained in the fluid.

4. Device according to claim 1, wherein the protection for the temporary receptors comprises a protective wrapper (18) configured for:
closing off the opening of the cover prior to placement of the sensor in the housing of the sensor holder, and
cooperating with the sensor holder so as to get perforated face to the opening of the cover when the sensor is placed in the housing of the sensor holder.

5. Device according to claim 4, wherein the protective wrapper is
a polymer film, for example a polyolefin, optionally a substituted one such as polytetrafluoroethylene (PTFE) or a silicone such as polydimethylsiloxane (PDMS); and/or
a film having a thickness comprised between 50 μm and 150 μm.

6. Device according to claim 4, wherein the opening of the cover has an inlet opening (16a) configured to admit fluid into the measurement chamber and an outlet opening (16b) configured to discharge fluid from the measurement chamber, and wherein, preferably, the protective wrapper comprises a first cap (180a) configured to close off the inlet opening prior to placement of the sensor in the housing of the sensor holder and a second cap (180b) configured to close off the outlet opening prior to placement of the sensor in the housing of the sensor holder.

7. Device according to claim 1, wherein the change in local property is a change of optical index in the sensor or a change of mass of a membrane (23) in the sensor.

8. Device according to claim 1, wherein the electronic chip is a photonic chip comprising at least one light guide (13) in which the temporary receptors are arranged, the light guide comprising a light input (135) and a light output (136), and wherein the transducer (130, 131) comprises:
a coherent light source (130) aligned with the light input of the light guide and capable of emitting a beam of coherent light into the light guide of the electronic chip;
an optical detector (131) aligned with the light output of the light guide and able to measure an optical parameter of the beam of coherent light exiting the light guide.

9. Device according to claim 1, wherein the electronic chip is an electromechanical chip comprising a membrane (23) on which the temporary receptors are arranged, and wherein the transducer (230, 231) comprises an actuator (230) capable of causing the membrane to vibrate, and a vibration frequency detector (231) for detecting the vibration frequency of the membrane.

10. Device according to claim 1, wherein the sensor holder comprises:
a positioning guide (52) for the sensor, configured to guide the sensor as it is placed in the housing of the sensor holder;
a retaining member (53) for holding the sensor in the housing, configured to hold the sensor in the housing when the sensor is placed in the housing of the sensor holder;
a connection socket (56a, 56b) configured to cooperate with the opening of the cover when the sensor is placed in the housing.

11. Device according to claim 10, wherein the opening of the cover and the connection socket are configured to allow the protective wrapper to get perforated face to the opening of the cover, and further wherein the protection for the temporary receptors comprises a protective wrapper (18) configured for:
closing off the opening of the cover prior to placement of the sensor in the housing of the sensor holder, and
cooperating with the sensor holder so as to pet perforated face to the opening of the cover when the sensor is placed in the housing of the sensor holder.

12. Consumable and interchangeable sensor (10) comprising an electronic chip (12) and a cover (15) integrally secured to the electronic chip, the electronic chip comprising a measurement chamber (11) comprising temporary receptors (14) capable of an interaction with an analyte (2) present in a fluid to be analyzed, the interaction causing a change in local property, and the cover comprising an opening (16a, 16b) suitable for admitting the fluid into the measurement chamber and for discharging the fluid from the measurement chamber,
the sensor being intended to be reversibly placed in a housing (51) of a sensor holder (50), and optionally comprising all or part of a transducer for the change in local property (130, 131; 230, 231) caused by the interaction between the temporary receptors and the analyte, the transducer being able to convert the change in local property into an electronic signal expressing the change in local property, and
the sensor comprises a protection (17) for the temporary receptors which is configured to be active prior to placement of the sensor in the housing of the sensor holder and configured to cooperate with the sensor holder so as to be deactivated by placement of the sensor in the housing of the sensor holder.

13. Sensor according to claim 12, wherein it comprises a protective wrapper (18) configured for:
closing off the opening of the cover prior to placement of the sensor in the housing of the sensor holder, and
cooperating with the sensor holder so as to get perforated face to the opening of the cover when the sensor is placed in the housing of the sensor holder.

14. Method of manufacturing a consumable and interchangeable sensor (10) according to claim 12, characterized in that it consists essentially of
i) preparing a silicon wafer;
ii) functionalizing the silicon wafer to form a plurality of electronic chips (12);
iii) introducing temporary receptors (14) onto a surface of the silicon wafer, on each electronic chip, to form measurement chambers (11);
iv) covering, with a protective layer, the surface of the silicon wafer onto which the temporary receptors have been introduced, the protective layer forming a cover (15) for each measurement chamber;
v) providing an opening (16a, 16b) on each cover to allow admitting a fluid into the measurement chamber associated with the cover and discharging the fluid from the measurement chamber associated with the cover;
vi) cutting the silicon wafer to separate each assembly formed by a measurement chamber and a cover;
vii) providing a protection (17) on the opening of each cover so as to form the sensors;
viii) collecting the sensors.

15. Method of manufacturing an electronic analysis device according to claim 1, characterized in that it essentially consists of
i) mass-producing consumable sensors (10);
ii) preferably, storing the mass-produced consumable sensors;
iii) separately producing sensor holders (50);
iv) assembling a consumable sensor with a sensor holder, preferably on a to-order basis.

16. Method according to claim 15, wherein the temporary receptors of the measurement chamber of the electronic analysis device are selected among molecules, peptides, polymers, biomarkers, nanoparticles or carbon nanotubes and further wherein the assembly step (iv) comprises:
iv.1) taking a consumable sensor;
iv.2) preparing a sensor holder, wherein the sensor holder comprises:
a positioning guide (52) for the sensor, configured to guide the sensor as it is placed in the housing of the sensor holder;
a retaining member (53) for holding the sensor in the housing, configured to hold the sensor in the housing when the sensor is placed in the housing of the sensor holder;
a connection socket (56a, 56b) configured to cooperate with the opening of the cover when the sensor is placed in the housing;
iv.3) positioning the consumable sensor relative to the sensor holder so that the opening of the cover is aligned with the connection socket (56a, 56b) of the sensor holder;
iv.4) reversibly placing the sensor in the housing (51) of the sensor holder;
iv.5) and, simultaneously or not simultaneously with step (iv.4), piercing the protective wrapper (18) facing the opening of the cover.
